(19) United States
(12) Patent Application Publication
(10) Pub. No.: US 2012/0171263 A1
(43) Pub. Date: Jul. 5, 2012

(54) Oil-In-Water Nanoemulsion Containing Brazil Nut Oil, As Well As Cosmetic Composition And Cosmetic Product Comprising Same And Use Of Said Nanoemulsion

(75) Inventors: Clarissa Capelas Romeu, Barueri (BR); Jean Luc Gesztesi, Sao Paulo (BR); Leandra Moraes Santos, Campinas (BR); Philip Leite Ribeiro, Sao Paulo (BR)

(73) Assignee: Natura Cosmeticos S.A., Itapecerica da Serra (BR)

(21) Appl. No.: 13/139,764

(22) PCT Filed: Dec. 17, 2009
(86) PCT No.: PCT/BR09/00413

§ 371 Date: Mar. 7, 2012

(30) Foreign Application Priority Data
Dec. 18, 2008 (FR) 0858766

(51) Int. Cl. A61K 8/97; A61Q 5/00; A61K 8/06; A61Q 5/12; B82B 1/00; B82Y 5/00

(52) U.S. Cl. 424/401; 424/70.11; 424/70.19; 424/70.17; 977/926; 977/773

(57) ABSTRACT

The present invention refers to an oil-in-water nanoemulsion comprising Brazil nut oil and an emulsifying system for cosmetic use, particularly for hair care. The emulsifying system comprises at least one non-ionic surfactant and at least one cationic component selected from a cationic surfactant and a cationic polymer. Another objective of the present invention is cosmetic compositions and cosmetic products that comprise a nanoemulsion characterized above.

The present invention refers to oil-in-water nanoemulsions comprising Brazil nut oil and an emulsifying system, cosmetic composition and cosmetic product comprising said nanoemulsions for hair care.

Emulsions are thermo-dynamically instable, heterogeneous systems, defined as a mixture of two immiscible liquids, one of which is dispersed in the other in the form of globules or droplets. The emulsions whose droplets are of an oil-in-water are referred to as O/W (oil-in-water) and emulsions with droplets of water in an oil are called W/O (water-in-oil). Classical emulsions or macroemulsions have droplets within the size range of 2 to 20 micrometers, conferring them a white or opaque appearance.

Nanoemulsions differ from macroemulsions by the smaller size of the oily particles, which are normally between 20 and 500 nanometers.

Nanoemulsions have been characterized as transparent or translucent depending on the size of the particles and the difference in the index of refraction between the oil and the aqueous phase. They are formulated using high pressure homogenizers.

### BACKGROUND OF THE INVENTION

The Brazilian patent application PI 0404595-5, filed by the same applicant, discloses an oil-in-water nanoemulsion for cosmetic use, wherein the oily particles have an average diameter varying between 50 and 200 nm (nanometers). The nanoemulsion comprises an emulsifying system having components such as ceteareth-20, ceteareth-12, glyceryl stearate, cetearyl alcohol and cetyl palmitate. This composition confers the nanoemulsion an opaque color, besides providing the skin the inherent properties of the nanoemulsion, such as improved absorption of the components by the skin, conferring softness, smoothness and hydration for 24 hours, and can also be added to products indicated for hair care. The composition of the nanoemulsion without addition of preservatives confers bactericide action. The nanoemulsion is used preferably for cosmetic applications for the body, face and hair, such as milk, lotions and gels. However, the oil-in-water nanoemulsions comprising Brazil nut oil and an emulsifying system, according to the present invention, have enhanced properties and performance in hair care.

Besides the document mentioned above, some relevant documents of the state of the art related to the subject matter of the present invention are listed below.

Document US 2004/157754 (corresponding to Brazilian patent application PI 0406785-1) discloses a composition for personal care containing about 5% by weight of a cationic cellulose polymer; from about 5 to about 50% by weight of an anionic surfactant system with a ethoxylate content and a sulphate content, wherein said ethoxylate content is in the amount of 1.04 multiplied by the molecular weight of said cationic cellulose polymer, divided by 1,000,000 and added to from about 0.75 to about 3.25, wherein said sulphate content is in the amount of 0.42 multiplied by the load density of said cationic cellulose polymer and added to from about 1.1 to about 3.6; from about 0.01 to about 5% by weight of a mono- or divalent salt; and at least about 20% by weight of an aqueous vehicle.

Document US 2004/151746 discloses a process for preparing a cationic emulsion and a cosmetic composition obtained by said process, besides the use of the composition. The preparation process comprises the following steps:

a) mix, with shaking, at least one fatty compound and at least one non-ionic surfactant, preferably at least two non-ionic surfactants at a temperature above the melting point of the fatty compound and of the non-ionic surfactant (between 20 and 85° C.), under normal atmospheric pressure (1 atm at sea level);

b) add water, with shaking;

c) add at least one cationic surfactant resulting in the cationic emulsion;

d) cool to ambient temperature (approximately 20° C.).

This document discloses the possibility of obtaining an oil-in-water nanoemulsion from this process. The nanoemulsion would then have particles smaller than 100 nm, preferably between 10 and 100 nm, preferably between 20 and 90 nm.

Document U.S. Pat. No. 6,039,936 (corresponding to Brazilian patent application PI 9705382-1) discloses an oil-in-water nanoemulsion that comprises oily globules smaller than 150 nm, an anphiphilic lipid non-ionic and at least one anphiphilic cationic lipid. Additionally, applications in cosmetics and dermopharmaceuticals are disclosed.

Document U.S. Pat. No. 6,375,960 (corresponding to Brazilian patent application PI 9906206-2) discloses a nanoemulsion that includes an oily phase dispersed in an aqueous phase and at least one solid surfactant at a temperature lower than or equal to 45° C. selected from groups that include ethoxylated fatty esters, ethoxylated fatty ethers and mixtures thereof. The process for preparing the nanoemulsion and methods for use thereof are also disclosed. The nanoemulsion obtained is transparent, stable, and is particularly used in topical pharmaceutical, ophthalmic and opthalmological compositions and cosmetics (applications for skin, hair, scalp, mucosae and eyes).

Document U.S. Pat. No. 6,375,960 (corresponding to Brazilian patent application PI 0100337-2) discloses an oil-in-water nanoemulsion whose oil globules are on average size smaller than 150 nm, comprising at least one oil, at least one anphiphilic lipid, preferably non-ionic or anionic, and at least one polyethylenoglycol ester or ether, as well as their uses in cosmetics or in dermopharmaceuticals.

Document U.S. Pat. No. 5,925,341 (corresponding to Brazilian patent application PI 9801355-6) discloses an oil-in-water emulsion whose oil globules are on average size smaller than 150 nm, comprising an anphiphilic lipid phase that comprises at least one non-ionic anphiphilic lipid, which is liquid at ambient temperature lower than 45° C., at least one oil and at least one silicone laminate, as well as their uses in cosmetics and dermopharmeuticals.

Document US 2002/032134 discloses a detergent and cosmetic compositions that comprise, in a cosmetically acceptable medium, a washing base and a conditioning system including an oily nanoemulsion. These compositions are indicated for application to the hair and to the skin. The nanoemulsion comprises oily globules smaller than 150 nm and an anphiphilic phase with at least one anphiphilic non-ionic lipid.

Document US 2003/087967 discloses a nanoemulsion that comprises an oily phase dispersed in an aqueous phase having oily globules smaller than 100 nm and comprises a ternary surfactant system with at least two non-ionic surfactants, such as ethoxylated fatty ester and sorbitan fatty acid ester, and at least one ionic surfactant, with alkali metal salts of cetyl phosphate and alkali metal salts of palmitoyl sarcosinate.

None of the documents of the state of the art, either those listed above or any other relating to compositions and processes relating to nanoemulsions considered less relevant for the present invention, is capable of disclosing the oil-in-water nanoemulsions of the present invention, that comprise Brazil nut oil and an emulsifying system, useful for hair care, as well as the process for preparation thereof disclosed in the present invention. Said nanoemulsions can be used to prepare a cosmetic composition and a cosmetic product, conferring them enhanced properties and performance in hair care, which result from a better external restructuring of the hair strands.

Persons skilled in the art know that the morphological structure of a strand of hair is composed of cuticles (outermost part), microfibrilles of the cortex (innermost part) and the cell membrane complex (CMC), which consists of membranes and adhesive material that joins the cuticles and the microfibrilles of the cortex. More recently, research in the field of cosmetics relating to hair treatment has sought similar compositions to the cell membrane complex, or part thereof, with the aim of uniting the cuticle cells of the hair filament.

The cosmetic composition and the cosmetic product of the present invention reduce the superficial damage of the hair by setting the raised cuticles, causing outer restructuring of the capillary fiber. The components used in the nanoemulsion of the present invention permit increased interaction with the negative sites of the hair, thus improving the contact with the capillary filament. The synergistic effect between the Brazil nut oil and the emulsifying system used in the present invention allows significantly better results to be achieved in terms of repair of the hair filaments in relation to the compositions of the state of the art.

The characteristics described above and the consequent advantages are comprised within the present invention and will now be described.

### BRIEF DESCRIPTION OF THE INVENTION

An objective of the present invention is an oil-in-water nanoemulsion, comprising Brazil nut oil and an emulsifying system for cosmetic use, particularly for hair care.

The emulsifying system comprises at least one non-ionic surfactant and at least one cationic component selected from at least one cationic surfactant and at least one cationic polymer.

Preferably, the non-ionic surfactant is one or more from Trideceth 12, Emulgade SE, Ceteareth-20, Sorbitan monolaurate and Polysorbate 80.

Preferably, the cationic surfactant is at least one from BTAC (behentrimonium chloride) and Dehyquart F75 (distearoylethyl hydroxyethylmonium methosulfate and cetearyl alcohol) and the cationic polymer is Polyquart H81 (PEG 15 cocopolyamine).

Another objective of the present invention is cosmetic compositions and cosmetic products that comprise the nanoemulsion characterized above.

### DETAILED DESCRIPTION OF THE INVENTION

According to the present invention, oil-in-water nanoemulsions were developed comprising Brazil nut oil and an emulsifying system for application in hair care.

The Brazil nut is the seed of the Bertholletia excelsa, a tree of the Lecythidaceae family. The Brazil nut oil is clear in color, almost transparent, and its taste is suave and agreeable, meaning it is especially used in the food industry. Fatty acids are present in Brazil nut oil in the following approximate contents: 17% of palmitic acid (C16:0), 0.5% of palmitoleic acid (C16:1), 10% of stearic acid (C18:0), 38% of oleic acid (C18:1) and 35% of linoleic acid (C18:2).

The present invention uses Brazil nut oil as liposoluble active substance in the internal phase of the droplets of an oil-in-water nanoemulsion in preparing a cosmetic composition and a cosmetic product which has enhanced properties and performance in hair care, resulting in a better external restructuring of the hair strands.

Nanoemulsions differ from macroemulsions by their smaller size of oily particles, which are normally between 20 and 500 nanometers.

Nanoemulsions have been characterized as transparent or trans-lucid depending on the size of the particles and the difference in the index of refraction between the oil and the aqueous phase. They are easily formulated using high pressure homogenenizers.

The present invention describes an oil-in-water nanoemulsion that has oily particles of a reduced average diameter varying between 100 and 280 nm.

A nanoemulsion comprises an emulsifying system that comprises at least one non-ionic surfactant and at least one cationic component selected from a cationic surfactant and a cationic polymer. Preferably, the non-ionic surfactant is one or more from Trideceth 12, Emulgade SE, Ceteareth-20, Sorbitan monolaurate and Polysorbate 80. Preferably, the cationic surfactant is at least one from BTAC (behentrimonium chloride) and Dehyquart F75 (distearoylethyl hydroxyethylmonium methosulfate and cetearyl alcohol) and the cationic polymer is Polyquart H81 (PEG 15 cocopolyamine).

Cosmetic compositions and cosmetic products that comprise a nanoemulsion of the present invention, besides providing the hair inherent properties of the nanoemulsion, such as improved absorption of the components, reduce the superficial damage of the strands by setting of the raised cuticles, causing external restructuring of the capillary fiber.

Non-limitative examples of cosmetic products that can be prepared from the nanoemulsion of the present invention are “rinse off” products, such as hair conditioner, and “leave on” products, such as hair spray or cream.

The nanoemulsions of the present invention present various advantages and characteristics desirable in a cosmetic product for hair care, some of which are listed below:

i) They are stable for a period of at least two years;

ii) They are characterized as kinetically stable systems, that is, they are physically stable for a longer period of time, verging on a thermodynamically stable system;

iii) They provide hair shine, smoothness and hydration, and assist with brushability and diffusion of the technology in the capillary fiber;

iv) They have no comedogenicity, phototoxicity and allergenicity;

v) They do not cause any kind of adverse reaction or cutaneous or occular lesion under normal use conditions;

vi) They are compatible with a broad range of active ingredients;

vii) The nanoemulsion of the present invention provides the skin softness, suaveness, hydration, non-stickiness, high penetration and spreadability, besides having excellent degree of homogeneity and stability;

viii) It can be used in sun protection products, “leave on” for hair, hair conditioners, ampoules for treating hair, among others.

Research carried out with hair dressers and consumers revealed that hair treated with a cosmetic product containing a nanoemulsion according to the present invention have greater smoothness and shine and the hair is freer, with more movement and easier to untangle with fingers and a comb. A reduction was noted in the ‘frizz’ effect, as well as a reduction in hair volume and enhanced brushability.

The synergistic effect between the Brazil nut oil and the emulsifying system used in the present invention allows the achievement of the significantly superior results mentioned above in relation to compositions of the state of the art. The components of the cationic nanoemulsion of the present invention allow greater interaction with the negative sites of the hair, thus improving the contact with the capillary filament. The increased stability of the nanoemulsion of the present invention is due to the steric impediment and the electrostatic repulsion caused by the cationic component.

Besides, the nanoemulsions of the present invention are characterized as release systems of lipophilic drugs, because the small size of its particles increases the contact area, the spreadability, the homogeneity of the distribution and the penetration of the active ingredient in the substrate (hair).

### Nanoemulsion of the Present Invention

The nanoemulsions of the present invention, preferably, comprise from 1% to 30% by weight of Brazil nut oil, more preferably from 4% to 20% by weight, based on the total weight of the nanoemulsion composition. They also comprise, preferably, from 5% to 12% by weight of an emulsifying system that comprises at least one non-ionic surfactant and at least one cationic component selected from a cationic surfactant and a cationic polymer.

The concentration of non-ionic surfactants in the composition of the nanoemulsion varies from around 2% to 4% by weight and the concentration of cationic components in the composition of the nanoemulsion varies from 3% to 8% by weight, based on the total weight of the nanoemulsion composition.

Preferably, the non-ionic surfactant is one or more from Trideceth 12, Emulgade SE, Ceteareth-20, Sorbitan monolaurate and Polysorbate 80. Preferably, the cationic surfactant is at least one from BTAC (behentrimoniurn chloride) and Dehyquart F75 (distearoylethyl hydroxyethylmonium methosulfate and cetearyl alcohol) and the cationic polymer is Polyquart H81 (PEG 15 cocopolyamine).

Therefore, the composition of the nanoemulsion of the present invention is constituted of an oily phase, which comprises Brazil nut oil and an emulsifying system as defined above, in a range of between 6% and 42% by weight, based on the total weight of the nanoemulsion composition.

### Fragrance

To compositions of this kind, it is optional to add perfume or fragrance selected from a range of possible substances. The quantity of fragrance in the oily phase to be added to the nanoemulsion of the present invention, preferably, varies, if present, from about 0.2% to about 6.0% by weight, based on the total weight of the nanoemulsion composition.

### Vehicle (Carrier)

Water is the basis of various possibilities of cosmetic compositions prepared from cosmetic composition base already described, acting as a vehicle for other components. The aqueous phase of the nanoemulsion of the present invention comprises water preferably demineralized or distilled in a suitable percentage (q.s.) to achieve 100% of the formula based on the total weight of the nanoemulsion composition.

### Other Optional Components

To provide the nanoemulsion of the present invention some desirable characteristic not achieved with the components already cited, it is possible to add optional components that are compatible with the properties thereof. Some such compounds that can be added to the composition are:
Other active principles: either lipophilic or hydrophilic, such as seaweed extracts, combination of palmitoyl hydroxypropyltrimonium aminopectin, glycerin crosspolymer, lecitin and grape seed oil, D-pantenol (conditioning agent), tocopherol (vitamin E), retinole (vitamin A), ascorbic acid (vitamin C), erocalcipherol (vitamin D) and sun filters commonly added to compositions of products for capillary use;
Bacteriostatics, bactericides or antimicrobials, such as Irgasan DP300;
Coloring agents;
Chelator agent, such as ethylenediaminetetraacetic acid (EDTA) and salts thereof;
pH adjusting agent, such as triethanolamine;
Preservative, such as DMDM hydantoin;
Plant extracts: chamomile, rosemary, thyme, calendula, carrot extract, juniper extract, gentian extract, cucumber extract;
Thickening agent: guar gum to make composition more viscous. In the absence of this gum, for example, the composition becomes quite liquid, fluid;
Anti-oxidant agents of lipophilic substances, such as butylated hydroxytoluene (BHT), butylated hydroxyanisole (BHA); and
Other cosmetically acceptable components that are compatible with the composition base.

### Examples of Composition

The following examples are preferred variations of nanoemulsion, object of the present invention, and should not be interpreted as being limitative of the invention. In this sense, it should be understood that the scope of the present invention encompasses other possible variations, being limited only by the content of the appended claims, possible equivalents being included therein.

| Components | Composition A | Composition B |
|---|---|---|
| Demineralized water | 62.1% | 85.23% |
| DMDM Hydantoin IPBC | 0.03% | 0.03% |
| Disodium EDTA | 0.1% | 0.1% |
| Ceteareth-20 | — | 0.36% |
| Brazil nut oil | 20.0% | 4.0% |
| Trideceth-12 | 4.0% | — |
| Behentrimonium chloride | 5.0% | 5.0% |
| Bidistillated glycerol | 5.0% | 5.0% |
| Emulgade SE-PF | — | 2.14% |
| TBHQ | 0.01% | 0.01% |
| Dehiquart F75 | 7.2% | — |
| 1% sodium hydroxide solution | 1.56% | — |

### Process for Preparing Nanoemulsion

The nanoemulsion that is the object of the present invention can be prepared using the process described below, the phases cited being:

| | |
|---|---|
| Phase A | Brazil nut oil |
| | Emulsifying system according to the present invention |
| Phase B | Water |
| Phase C | Cooling water |

### Step I. Preparing the Emulsion

a. Mix the Brazil nut oil and the emulsifying system (phase A) in an auxiliary equipment and heat to 75° C.;

b. Add water (phase B) to the main equipment and heat to 75° C.;

c. Add phase A to the main equipment and shake at 1500 rpm for 30 minutes keeping the temperature between 70° C.-75° C.; and

d. Add cooling water (phase C) and shake for 15 minutes at 1500 rpm;

Optionally, substep e can also be carried out, as follows:

e. Add components such as, for example, fragrance, preservative, coloring agents, hydrophilic active ingredients, among others to the composition of step d.

The shaking referred to above preferably should be in rotor-stator homogenizers.

Step II. Preparing the Nanoemulsion from the Prepared Emulsion

a. Transfer the composition obtained in Step I to a high pressure equipment with preferably from one to three pistons. This equipment has two chambers, the first chamber having a pressure of 120 MPa (1200 bar) and the second chamber having a pressure of 12 MPa (120 bar);

b. The nanoemulsion of the present invention is automatically transferred from the first chamber at a pressure of 120 MPa (1200 bar) to the second chamber at a pressure of 12 MPa (120 bar), concluding a cycle.

c. To obtain the average size of particles between 100 and 280 nm, repeat if necessary the substep above, totaling three cycles.

Alternatively, it is possible to use different numbers of cycles, if different particle sizes are desired.

The passage of the emulsion through this difference in pressure (depressurization) causes the explosion of drops of oil and the collision thereof against walls of the second chamber. With this, the particles thereafter have a diameter in the order of nanometers. To obtain an average size particle of around 100 to 280 nm, the emulsion must undergo one to three times sequentially in this equipment.

This is one of the most simple processes known in the art, since a single passage through the equipment described above is required. Techniques to prepare the nanoemulsion of the state of the art, normally recommend the use of more cycles and do not express the need for depressurization (120 MPa-1200 bar to 12 MPa-120 bar) to decrease the particle size to the order of nanometers.

### Example of Process for Preparing Nanoemulsion

To exemplify the process, the composition of nanoemulsion described in example 1 above will be used.

| | |
|---|---|
| Phase A | Emulgin B2 |
| | Emulgade ® SE |
| | Cupuaçu butter |
| | Behentrimonium |
| | Phenyl trimethicone |
| | BHT |
| | BHA |
| Phase B | Demineralized Water |
| | Disodium EDTA |
| | Vegetable glycerin |
| | Biossaccharide gum-1 |
| Phase C | Demineralized cooling water |
| Phase D | Essence Comfort 404 |
| Phase E | Orange Solution K 7011 |
| | Blue Solution |
| | Red Solution 0.2% |
| | Yellow Solution |
| Phase F | Triethanolamine |
| Phase G | DMDM Hydantoin/IPBC |

### Step I

a. Add the components of phase A in an auxiliary equipment and heat to 75° C.;

b. Add the components of phase B in a main equipment and heat to 75° C.;

c. Add a phase A in main equipment, shaking at approximately 1500 rpm for 30 minutes keeping the temperature between 70° C.-75° C.;

d. Add cooling water (phase C) and shake for 15 minutes at 1500 rpm;

e. Add phase D and shake for 5 minutes;

f. Add phase E and shake for 5 minutes;

g. Add phase F and shake for 5 minutes;

h. Add phase G and shake for 5 minutes;

### Step II

Insert the emulsion obtained in step (h) in the high pressure equipment and the product will pass through two chambers: the first chamber under a pressure of 120 MPa (1200 bar) and the second chamber under a pressure of 12 MPa (120 bar), totaling a cycle to obtain the average size of particles of 50 to 200 nm.

The equipment used is the PANDA (equipment with 1 piston—laboratory scale), Panter or Tiger (equipment with three pistons—industrial scale 50 or 1000 liters/hour), which works as follows: the product arrives at the first chamber by gravity. The first chamber attains the pressure chosen during research into the technology, that is, the second chamber represents 10% of the first chamber. The product is pressured through a hole to attain the second chamber. Depressurization and also the impact of the particles on the walls of the second chamber lead to a reduction in particle size to the order of nanometers.

### Safety Tests Performed:

Safety tests were performed (Pc5, soap chamber and acceptability) with eight different prototypes presenting different concentrations of Brazil nut oil.

### Technology 1

### Pc5, SOAP CHAMBER AND ACCEPTABILITY

Prototype 1 (20% oil)
Prototype 2 (4% oil)
Prototype 3 (4% oil)
Prototype 4 (4% oil—dilution of the prototype 1)

### Technology 2

### PC5 E SOAP CHAMBER

Prototype 5 (4% oil)
Prototype 6 (4% oil)
Prototype 7 (20% oil)
Prototype 8 (4% oil—dilution of the prototype 7)

Pc5: all the prototypes presented very good cutaneous compatibility Soap Chamber: all the prototypes present results of Tewi and irritation index lower than those of the positive control and, therefore, coherent with its respective product category, in the effect on the cutaneous barrier

Acceptability: the prototypes of technology 1 proved safe for use in humans, in the area recommended by the manufacturer, causing no reactions of irritation or sensitivity in the analyzed sample of volunteers.

1. Oil-in-water nanoemulsion for cosmetic use in hair care, comprising, in an oily phase, Brazil nut oil and an emulsifying system comprising at least one non-ionic surfactant and at least one cationic component selected from at least one cationic surfactant or at least one cationic polymer.

2. Nanoemulsion according to claim 1, wherein said at least one non-ionic surfactant is selected from Trideceth 12, Emulgade SE, Ceteareth-20, Sorbitan monolaurate and Polysorbate 80.

3. Nanoemulsion according to claim 1, wherein said cationic surfactant is at least one from BTAC (behentrimonium chloride) and Dehyquart F75 (distearoylethyl hydroxyethylmonium methosulfate and cetearyl alcohol).

4. Nanoemulsion according to claim 1, wherein said cationic polymer is Polyquart H81 (PEG 15 cocopolyamine).

5. Nanoemulsion according to claim 1, wherein said oily phase has particles with an average diameter of 100 to 280 nm.

6. Nanoemulsion according to claim 1, comprising from 1% to 30% by weight of Brazil nut oil, based on the total weight of the nanoemulsion composition.

7. Nanoemulsion according to claim 6, comprising from 4 to 20% by weight of Brazil nut oil, based on the total weight of the nanoemulsion composition.

8. Nanoemulsion according to claim 1, comprising from 5% to 12% by weight of the emulsifying system, based on the total weight of the nanoemulsion composition.

9. Nanoemulsion according to claim 1, comprising from 2% to 4% by weight of said at least one non-ionic surfactant, based on the total weight of the nanoemulsion composition.

10. Nanoemulsion according to claim 1, comprising from 3% to 8% by weight of said at least one cationic component, based on the total weight of the nanoemulsion composition.

11. Nanoemulsion according to claim 1, comprising from 6% to 42% by weight of said oily phase, based on the total weight of the nanoemulsion composition.

12. Cosmetic composition for hair care, comprising a nanoemulsion as defined in claim 1.

13. Cosmetic product for hair care, comprising a composition as defined in claim 12.

14. Cosmetic product according to claim 13, wherein the product is in the form of a “rinse off” type product.

15. Cosmetic product according to claim 13, wherein the product is in the form of a hair conditioner.

16. Cosmetic product according to claim 13, wherein the product is in the form of a “leave on” type product.

17. Cosmetic product according to claim 16, wherein the product is in the form of a hair spray or cream.

18. Use of nanoemulsion as defined in claim 1 for manufacturing a cosmetic composition indicated for hair care.
